(19) United States
(12) Patent Application Publication
(10) Pub. No.: US 2023/0119002 A1
(43) Pub. Date: Apr. 20, 2023

(54) COMPOSITIONS AND METHODS CONTAINING REDUCED NICOTINAMIDE RIBOSIDE FOR PREVENTION AND TREATMENT OF CARDIOVASCULAR DISEASES AND CONDITIONS

(71) Applicant: SOCIETE DES PRODUITS NESTLE S.A., Vevey (CH)

(72) Inventors: CARLES CANTO ALVAREZ, Chicago, IL (US); STEFAN CHRISTEN, Ecublens (CH); MARIA PILAR GINER, Pully (CH); JUDITH GIROUD-GERBETANT, Igualada Catalunya (ES); SOFIA MOCO, Amsterdam (NL); PHILIPP GUT, Geneve (CH); MAGALI JOFFRAUD, Ecublens (CH); JOY RICHARD, Bretigny-sur-Morrens (CH)

(21) Appl. No.: 17/905,210

(22) PCT Filed: Mar. 9, 2021
(86) PCT No.: PCT/EP2021/055937

§ 371 Date: Aug. 29, 2022

(30) Foreign Application Priority Data
Mar. 9, 2020 (EP) 20161849.3

(51) Int. Cl. A61K 31/706; A61P 9/00

(52) U.S. Cl. CPC A61K 31/706; A61P 9/00

(57) ABSTRACT

The present invention provides compounds and compositions containing reduced nicotinamide riboside for use in methods of prevention and/or treatment of cardiovascular disease and/or conditions. In one embodiment of the invention, said compounds and compositions of the invention maintain or improve cardiovascular tissue and cellular function. In another embodiment of the invention, said compounds and compositions of the invention enhance cardiovascular recovery after injury or surgery.

### FIELD OF THE INVENTION

The present invention provides compounds and compositions containing reduced nicotinamide riboside for use in methods of prevention and/or treatment of cardiovascular disease and/or conditions. In one embodiment of the invention, said compounds and compositions of the invention maintain or improve cardiovascular tissue and cellular function. In another embodiment of the invention, said compounds and compositions of the invention enhance cardiovascular recovery after injury or surgery.

### BACKGROUND TO THE INVENTION

Cardiovascular disease refers to diseases of the circulatory system including the heart and blood vessels. They are one of the leading causes of death world-wide with major risk factors including high blood pressure, high circulating lipid levels, obesity and diabetes.

Nicotinamide adenine dinucleotide (NAD+) and related metabolites are central mediators of fuel oxidation and bioenergetics within cardiomyocytes. Additionally, NAD+ is required for the activity of multifunctional enzymes, including sirtuins and poly(ADP-ribose) polymerases that regulate posttranslational modifications, DNA damage responses, and Ca2+ signaling (Matasic et al. 2018)

Dysfunction in the myocardial NAD+ pool may be linked to metabolic remodeling and mitochondrial dysfunction in the failing heart. Stabilizing the intracellular NAD+ level represents a way to improve myocardial bioenergetics and cardiac function.

Therefore, there is an urgent unmet need to address cardiovascular diseases and/or conditions with new compounds, compositions and methods of prevention and/or treatment which influence NAD+.

### SUMMARY OF THE INVENTION

The present invention provides compounds and compositions for use in methods of prevention and/or treatment of cardiovascular conditions and diseases.

In an embodiment, the composition is selected from the group consisting of: a food or beverage product, a food supplement, an oral nutritional supplement (ONS), a medical food, and combinations thereof.

In another embodiment, the present invention provides a method for increasing intracellular nicotinamide adenine dinucleotide (NAD⁺) in a subject, the method comprising administering a compound or composition of the invention consisting of administering a reduced nicotinamide riboside to the subject in an amount effective to increase NAD⁺ biosynthesis.

In a further embodiment, as a precursor of NAD+ biosynthesis, reduced nicotinamide riboside, can increase in NAD+ biosynthesis and provide one or more benefits to cardiovascular function.

In another embodiment, the present invention provides a unit dosage form of a composition consisting of reduced nicotinamide riboside, the unit dosage form contains an effective amount of the reduced nicotinamide riboside to increase NAD+ biosynthesis.

In one embodiment of the invention, the composition containing reduced nicotinamide riboside is provided to maintain or increase cardiovascular function in a subject.

In yet another embodiment of the invention, the composition containing reduced nicotinamide riboside is provided to enhance recovery of the cardiovascular system after injury or surgery.

In another embodiment of the invention, the composition is a nutritional composition selected from a: food or beverage product, including food additives, food ingredients, functional foods, dietary supplements, medical foods, nutraceuticals, oral nutritional supplements (ONS) or food supplements.

In another embodiment of the invention, the composition is a nutritional composition containing reduced nicotinamide riboside wherein said composition is provided to prevent or treat cardiovascular diseases or conditions in a subject.

### DETAILED DESCRIPTION OF THE INVENTION

Definitions

All percentages expressed herein are by weight of the total weight of the composition unless expressed otherwise. As used herein, “about,” “approximately” and “substantially” are understood to refer to numbers in a range of numerals, for example the range of −10% to +10% of the referenced number, preferably −5% to +5% of the referenced number, more preferably −1% to +1% of the referenced number, most preferably −0.1% to +0.1% of the referenced number.

All numerical ranges herein should be understood to include all integers, whole or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in this invention and the appended claims, the singular forms “a,” “an” and “the” include plural referents unless the context clearly dictates otherwise. Thus, for example, reference to “a component” or “the component” includes two or more components.

The words “comprise,” “comprises” and “comprising” are to be interpreted inclusively rather than exclusively. Likewise, the terms “include,” “including” and “or” should all be construed to be inclusive, unless such a construction is clearly prohibited from the context. Nevertheless, the compositions disclosed herein may lack any element that is not specifically disclosed herein. Thus, a disclosure of an embodiment using the term “comprising” includes a disclosure of embodiments “consisting essentially of” and “consisting of” the components identified. Any embodiment disclosed herein can be combined with any other embodiment disclosed herein.

Where used herein, the terms “example” and “such as,” particularly when followed by a listing of terms, are merely exemplary and illustrative and should not be deemed to be exclusive or comprehensive. As used herein, a condition “associated with” or “linked with” another condition means the conditions occur concurrently, preferably means that the conditions are caused by the same underlying condition, and most preferably means that one of the identified conditions is caused by the other identified condition.

The terms “food,” “food product” and “food composition” mean a product or composition that is intended for ingestion by an individual such as a human and provides at least one nutrient to the individual. A food product typically includes at least one of a protein, a lipid, a carbohydrate and optionally includes one or more vitamins and minerals. The term “beverage” or “beverage product” means a liquid product or liquid composition that is intended to be ingested orally by an individual such as a human and provides at least one nutrient to the individual.

The compositions of the present disclosure, including the many embodiments described herein, can comprise, consist of, or consist essentially of the elements disclosed herein, as well as any additional or optional ingredients, components, or elements described herein or otherwise useful in a diet.

As used herein, the term “isolated” means removed from one or more other compounds or components with which the compound may otherwise be found, for example as found in nature. For example, “isolated” preferably means that the identified compound is separated from at least a portion of the cellular material with which it is typically found in nature. In an embodiment, an isolated compound is free from any other compound.

“Prevention” includes reduction of risk, incidence and/or severity of a condition or disorder. The terms “treatment,” “treat” and “to alleviate” include both prophylactic or preventive treatment (that prevent and/or slow the development of a targeted pathologic condition or disorder) and curative, therapeutic or disease-modifying treatment, including therapeutic measures that cure, slow down, lessen symptoms of, and/or halt progression of a diagnosed pathologic condition or disorder; and treatment of patients at risk of contracting a disease or suspected to have contracted a disease, as well as patients who are ill or have been diagnosed as suffering from a disease or medical condition. The term does not necessarily imply that a subject is treated until total recovery. The terms “treatment” and “treat” also refer to the maintenance and/or promotion of health in an individual not suffering from a disease but who may be susceptible to the development of an unhealthy condition. The terms “treatment,” “treat” and “to alleviate” are also intended to include the potentiation or otherwise enhancement of one or more primary prophylactic or therapeutic measure. The terms “treatment,” “treat” and “to alleviate” are further intended to include the dietary management of a disease or condition or the dietary management for prophylaxis or prevention a disease or condition. A treatment can be patient- or doctor-related.

The term “unit dosage form,” as used herein, refers to physically discrete units suitable as unitary dosages for human and animal subjects, each unit containing a predetermined quantity of the composition disclosed herein in an amount sufficient to produce the desired effect, in association with a pharmaceutically acceptable diluent, carrier or vehicle. The specifications for the unit dosage form depend on the particular compounds employed, the effect to be achieved, and the pharmacodynamics associated with each compound in the host.

As used herein, an “effective amount” is an amount that prevents a deficiency, treats a disease or medical condition in an individual, or, more generally, reduces symptoms, manages progression of the disease, or provides a nutritional, physiological, or medical benefit to the individual. The relative terms “improve,” “increase,” “enhance,” “promote” and the like refer to the effects of the composition disclosed herein, namely a composition comprising reduced nicotinamide riboside, relative to a composition not having nicotinamide riboside but otherwise identical. As used herein, “promoting” refers to enhancing or inducing relative to the level before administration of the composition disclosed herein.

As used herein “reduced nicotinamide riboside” may also be known as protonated nicotinamide riboside, dihydronicotinamide riboside, dihydro-1-beta-D-ribofuranosyl-3-pyridinecarboxamide, or 1-(beta-D-ribofuranosyl)-dihydronicotinamide. A description of the synthesis of reduced nicotinamide riboside is given in Example 1. The location of the protonation site can give rise to different forms of “reduced nicotinamide riboside”. For example: 1,4-dihydro-1-beta-D-ribofuranosyl-3-pyridinecarboxamide; 1,2-dihydro-1-beta-D-ribofuranosyl-3-pyridinecarboxamide; and 1,6-dihydro-1-beta-D-ribofuranosyl-3-pyridinecarboxamide (Makarov and Migaud, 2019).

Cardiovascular Diseases and Conditions

Cardiovascular diseases (CVDs) are a group of disorders of the heart and blood vessels which include:

(i) Coronary heart disease involving the blood vessels supplying the heart muscle;

(ii) Cerebrovascular disease involving blood vessels supplying the brain;

(iii) Peripheral arterial disease involving blood vessels supplying the arms and legs;

(iv) Rheumatic heart disease leading to damage to the heart muscle and heart valves from rheumatic fever, caused by streptococcal bacteria; and other inflammatory heart conditions;

(v) Congenital heart disease due to malformations of heart structure existing at birth;

(vi) Deep vein thrombosis and pulmonary embolism from blood clots in the leg veins, which can dislodge and move to the heart and lungs.

(i) Coronary Heart Disease—Disease of the Blood Vessels Supplying the Heart Muscle;

Coronary artery disease is caused by atherosclerosis which is the narrowing and/or blockage of the blood vessels that supply the heart. It is one of the most common forms of heart disease and the leading cause of myocardial infarction and angina.

It may be appreciated that the compounds, compositions and methods of the present invention may be beneficial to prevent and/or treat coronary artery disease and associated conditions.

(ii) Cerebrovascular Disease—Disease of the Blood Vessels Supplying the Brain;

Cerebral vascular disease is also caused by atherosclerosis which is the narrowing and/or blockage of the blood vessels that flow to the brain. If the flow of blood is cut off this can lead to strokes and transient ischemic attacks. A stroke occurs when the blood supply to the brain is interrupted. The brain is thus deprived of oxygen may be permanently damaged. Transient ischemic attacks when there is a brief blockage. The temporary loss of blood to the brain disappears quickly and permanent damage is unlikely.

It may be appreciated that the compounds, compositions and methods of the present invention may be beneficial to prevent and/or treat cerebrovascular disease and associated conditions.

(iii) Peripheral Arterial Disease—Disease of Blood Vessels Supplying the Arms and Legs;

Peripheral arterial disease (PAD) is caused by atherosclerosis, which is the narrowing and/or blockage of the blood vessels in the arms or legs. It may also manifest as an aneurysm which is a bulge or weakness in the wall of a blood vessel. Aneurysms can enlarge over time and may be life threatening if they rupture. They can occur because of high blood pressure or a weak spot in a blood vessel wall. Aneurysms can occur in arteries in any location in your body. The most common sites include the abdominal aorta and the arteries at the base of the brain.

It may be appreciated that the compounds, compositions and methods of the present invention may be beneficial to prevent and/or treat peripheral arterial disease and associated conditions.

(iv) Rheumatic Heart Disease and Other Inflammatory Heart Conditions

Rheumatic Heart Disease

Rheumatic heart disease is caused by damage to the heart valves and heart muscle from the inflammation and scarring caused by rheumatic fever. Rheumatic fever is caused by an abnormal response of the body to infection with streptococcal bacteria.

Other inflammatory heart diseases include: inflammation of the heart muscle (myocarditis), the membrane sac (pericarditis) which surround the heart, the inner lining of the heart (endocarditis) or the myocardium (heart muscle).

Inflammatory heart disease includes: cardiomyopathy, pericardial disease, and valvular heart disease.

It may be appreciated that the compounds, compositions and methods of the present invention may be beneficial to prevent and/or treat inflammatory heart diseases and associated conditions.

Cardiomyopathy

Cardiomyopathy refers to diseases of the heart muscle itself. Some types of cardiomyopathy are genetic, while others occur because of infection or other reasons that are less well understood. One of the most common types of cardiomyopathy is idiopathic dilated cardiomyopathy, where the heart is enlarged. Other types include ischemic, loss of heart muscle; dilated, heart enlarged; hypertrophic, heart muscle is thickened.

Pericardial Disease

The sac that encases the heart is called the pericardium and it can be affected by a variety of conditions such as inflammation (pericarditis), fluid accumulation (pericardial effusion) and stiffness (constrictive pericarditis).

Valvular Heart Disease

The heart's valves keep blood flowing through the heart in the right direction. But a variety of conditions can lead to valvular damage. Valves may narrow (stenosis), leak (regurgitation or insufficiency) or not close properly (prolapse). These diseases may be congenital or related to rheumatic fever, infections connective tissue disorders, and certain medications or radiation treatments for cancer.

It may be appreciated that the compounds, compositions and methods of the present invention may be beneficial to prevent and/or treat inflammatory heart diseases and associated conditions such as cardiomyopathy, pericardial disease or valvular heart disease.

(v) Congenital Heart Disease—Malformations of Heart Structure Existing at Birth;

A congenital heart disease is a problem which is associated with defects of the structure of the heart which is present at birth. Congenital heart defects typically involve the walls of the heart, the valves of the heart, and the arteries and veins near the heart.

It may be appreciated that the compounds, compositions and methods of the present invention may be beneficial for associated diseases and conditions related to congenital heart defects.

(vi) Deep Vein Thrombosis and Pulmonary Embolism—Blood Clots in the Leg Veins, which can Dislodge and Move to the Heart and Lungs.

Deep vein thrombosis, or DVT, is a blood clot that forms in a vein deep in the body. Most deep vein clots occur in the lower leg or thigh. If the vein swells, the condition is called thrombophlebitis. A deep vein thrombosis can break loose and cause a serious problem in the lung, called a pulmonary embolism.

It may be appreciated that the compounds, compositions and methods of the present invention may be beneficial to prevent and/or treat diseases and/or conditions associated with deep vein thrombosis and pulmonary embolism.

Recovery After Cardiovascular Injury and Surgery

The cardiovascular conditions and diseases mentioned above may require intervention by surgery. It may be appreciated that the compounds, compositions and methods of the present invention may be beneficial to for recovery after surgery and/or trauma to the cardiovascular tissue.

### Embodiments

The present invention provides compounds and compositions containing reduced nicotinamide riboside. Another aspect of the present invention is a unit dosage form of a composition consisting of reduced nicotinamide riboside, and the unit dosage form contains the reduced nicotinamide riboside in an amount effective to increase intracellular NAD in subject in need thereof.

The increase in NAD biosynthesis can provide one or more benefits to the individual, for example a human (e.g., a human undergoing medical treatment), a pet or a horse (e.g., a pet or horse undergoing medical treatment), or cattle or poultry (e.g., cattle or poultry being used in agriculture) with respect to prevention or treatment of cardiovascular disease.

For non-human mammals such as rodents, some embodiments comprise administering an amount of the composition that provides 1.0 mg to 1.0 g of the reduced nicotinamide riboside/kg of body weight of the non-human mammal, preferably 10 mg to 500 mg of the reduced nicotinamide riboside/kg of body weight of the non-human mammal, more preferably 25 mg to 400 mg of the reduced nicotinamide riboside/kg of body weight of the mammal, most preferably 50 mg to 300 mg of the reduced nicotinamide riboside/kg of body weight of the non-human mammal.

For humans, some embodiments comprise administering an amount of the composition that provides 1.0 mg to 10.0 g of the reduced nicotinamide riboside/kg of body weight of the human, preferably 10 mg to 5.0 g of the reduced nicotinamide riboside/kg of body weight of the human, more preferably 50 mg to 2.0 g of the reduced nicotinamide riboside/kg of body weight of the human, most preferably 100 mg to 1.0 g of the reduced nicotinamide riboside/kg of body weight of the human.

In some embodiments, at least a portion of the reduced nicotinamide riboside is isolated from natural plant sources. Additionally or alternatively, at least a portion of reduced nicotinamide riboside can be chemically synthesized. For example, according to Example 1 described below.

As used herein, a “composition consisting essentially of reduced nicotinamide riboside” contains reduced nicotinamide riboside and does not include, or is substantially free of, or completely free of, any additional compound that affects NAD+production other than the “reduced nicotinamide riboside”. In a particular non-limiting embodiment, the composition consists of the reduced nicotinamide riboside and an excipient or one or more excipients.

In some embodiments, the composition consisting essentially of reduced nicotinamide riboside is optionally substantially free or completely free of other NAD+precursors, such as nicotinamide riboside.

As used herein, “substantially free” means that any of the other compounds present in the composition is no greater than 1.0 wt. % relative to the amount of reduced nicotinamide riboside, preferably no greater than 0.1 wt. % relative to the amount of reduced nicotinamide riboside, more preferably no greater than 0.01 wt. % relative to the amount of reduced nicotinamide riboside, most preferably no greater than 0.001 wt. % relative to the amount of reduced nicotinamide riboside.

Another aspect of the present invention is a method for increasing intracellular NAD⁺ in a mammal in need thereof, comprising administering to the mammal a composition consisting essentially of or consisting of reduced nicotinamide riboside in an amount effective to increase NAD⁺ biosynthesis. The method can promote the increase of intracellular levels of NAD⁺ in cells and tissues for improving cell and tissue survival and overall cell and tissue health, for example, in cardiovascular cells and tissues, especially cardiomyocytes and tissues.

Nicotinamide adenine dinucleotide (NAD+) is considered a coenzyme, and essential cofactor in cellular redox reactions to produce energy. It plays critical roles in energy metabolism, as the oxidation of NADH to NAD+ facilitates hydride-transfer, and consequently ATP generation through mitochondrial oxidative phosphorylation. It also acts as a degradation substrate for multiple enzymes (Canto, C. et al. 2015; Imai, S. et al. 2000; Chambon, P. et al. 1963; Lee, H. C. et al. 1991).

Mammalian organisms can synthesize NAD+ from four different sources. First, NAD+ can be obtained from tryptophan through the 10-step de novo pathway. Secondly, Nicotinic acid (NA) can also be transformed into NAD+ through the 3-step Preiss-Handler path, which converges with the de novo pathway. Thirdly, intracellular NAD+ salvage pathway from nicotinamide (NAM) constitutes the main path by which cells build NAD+, and occurs through a 2-step reaction in which NAM is first transformed into NAM-mononucleotide (NMN) via the catalytic activity of the NAM-phosphoribosyltransferase (NAMPT) and then converted to NAD+ via NMN adenylyltransferase (NMNAT) enzymes. Finally, Nicotinamide Riboside (NR) constitutes yet a fourth path to NAD+, characterized by the initial phosphorylation of NR into NMN by NR kinases (NRKs)(Breganowski, P. et al.; 2004).

An important difference between NR and NRH is that they go through different synthetic pathways to synthesis NAD+. For example, NRH does not use the NRK-1 enzyme pathway (J. Giroud-Gerbetant et al. 2019). Therefore, their actions on NAD+are independent and unrelated.

Five molecules previously have been known to act as direct extracellular NAD+ precursors: tryptophan, nicotinic acid (NA), nicotinamide (NAM), nicotinic acid riboside (NaR) and nicotinamide riboside (NR). The present invention, discloses a new molecule that can act as an extracellular NAD+ precursor, reduced nicotinomide riboside (NRH). The reduction of the NR molecule to NRH confers it not only a much stronger capacity to increase intracellular NAD+ levels, but also a different selectivity in terms of its cellular use.

The present invention relates to NRH, a new molecule which can act as an NAD+ precursor. This reduced form of NR, which displays an unprecedented ability to increase NAD+ and has the advantage of being more potent and faster than nicotinamide riboside (NR). NRH utilizes a different pathway than NR to synthesize NAD+, which is NRK independent. The present invention demonstrates that NRH is protected against degradation in plasma and can be detected in circulation after oral administration. These advantages of the invention support its therapeutic efficacy.

The method comprises administering an effective amount of a composition consisting essentially of reduced nicotinamide riboside or consisting of reduced nicotinamide riboside to the individual.

In each of the compositions and methods disclosed herein, the composition is preferably a food product or beverage product, including food additives, food ingredients, functional foods, dietary supplements, medical foods, nutraceuticals, oral nutritional supplements (ONS) or food supplements.

The composition can be administered at least one day per week, preferably at least two days per week, more preferably at least three or four days per week (e.g., every other day), most preferably at least five days per week, six days per week, or seven days per week. The time period of administration can be at least one week, preferably at least one month, more preferably at least two months, most preferably at least three months, for example at least four months. In some embodiments, dosing is at least daily; for example, a subject may receive one or more doses daily, in an embodiment a plurality of doses per day. In some embodiments, the administration continues for the remaining life of the individual. In other embodiments, the administration occurs until no detectable symptoms of the medical condition remain. In specific embodiments, the administration occurs until a detectable improvement of at least one symptom occurs and, in further cases, continues to remain ameliorated.

The compositions disclosed herein may be administered to the subject enterally, e.g., orally, or parenterally. Non-limiting examples of parenteral administration include intravenously, intramuscularly, intraperitoneally, subcutaneously, intraarticularly, intrasynovially, intraocularly, intrathecally, topically, and inhalation. As such, non-limiting examples of the form of the composition include natural foods, processed foods, natural juices, concentrates and extracts, injectable solutions, microcapsules, nano-capsules, liposomes, plasters, inhalation forms, nose sprays, nosedrops, eyedrops, sublingual tablets, and sustained-release preparations.

The compositions disclosed herein can use any of a variety of formulations for therapeutic administration. More particularly, pharmaceutical compositions can comprise appropriate pharmaceutically acceptable carriers or diluents and may be formulated into preparations in solid, semi-solid, liquid or gaseous forms, such as tablets, capsules, powders, granules, ointments, solutions, suppositories, injections, inhalants, gels, microspheres, and aerosols. As such, administration of the composition can be achieved in various ways, including oral, buccal, rectal, parenteral, intraperitoneal, intradermal, transdermal, and intratracheal administration. The active agent may be systemic after administration or may be localized by the use of regional administration, intramural administration, or use of an implant that acts to retain the active dose at the site of implantation.

In pharmaceutical dosage forms, the compounds may be administered as their pharmaceutically acceptable salts. They may also be used in appropriate association with other pharmaceutically active compounds. The following methods and excipients are merely exemplary and are in no way limiting.

For oral preparations, the compounds can be used alone or in combination with appropriate additives to make tablets, powders, granules or capsules, for example, with conventional additives, such as lactose, mannitol, corn starch or potato starch; with binders, such as crystalline cellulose, cellulose functional derivatives, acacia, corn starch or gelatins; with disintegrators, such as corn starch, potato starch or sodium carboxymethylcellulose; with lubricants, such as talc or magnesium stearate; and if desired, with diluents, buffering agents, moistening agents, preservatives and flavoring agents.

The compounds can be formulated into preparations for injections by dissolving, suspending or emulsifying them in an aqueous or non-aqueous solvent, such as vegetable or other similar oils, synthetic aliphatic acid glycerides, esters of higher aliphatic acids or propylene glycol; and if desired, with conventional, additives such as solubilizers, isotonic agents, suspending agents, emulsifying agents, stabilizers and preservatives.

The compounds can be utilized in an aerosol formulation to be administered by inhalation. For example, the compounds can be formulated into pressurized acceptable propellants such as dichlorodifluoromethane, propane, nitrogen and the like.

Furthermore, the compounds can be made into suppositories by mixing with a variety of bases such as emulsifying bases or water-soluble bases. The compounds can be administered rectally by a suppository. The suppository can include a vehicle such as cocoa butter, carbowaxes and polyethylene glycols, which melt at body temperature, yet are solidified at room temperature.

Unit dosage forms for oral or rectal administration such as syrups, elixirs, and suspensions may be provided wherein each dosage unit, for example, teaspoonful, tablespoonful, tablet or suppository, contains a predetermined amount of the composition. Similarly, unit dosage forms for injection or intravenous administration may comprise the compounds in a composition as a solution in sterile water, normal saline or another pharmaceutically acceptable carrier, wherein each dosage unit, for example, mL or L, contains a predetermined amount of the composition containing one or more of the compounds.

Compositions intended for a non-human animal include food compositions to supply the necessary dietary requirements for an animal, animal treats (e.g., biscuits), and/or dietary supplements. The compositions may be a dry composition (e.g., kibble), semi-moist composition, wet composition, or any mixture thereof. In one embodiment, the composition is a dietary supplement such as a gravy, drinking water, beverage, yogurt, powder, granule, paste, suspension, chew, morsel, treat, snack, pellet, pill, capsule, tablet, or any other suitable delivery form. The dietary supplement can comprise a high concentration of the UFA and NORC, and B vitamins and antioxidants. This permits the supplement to be administered to the animal in small amounts, or in the alternative, can be diluted before administration to an animal. The dietary supplement may require admixing, or can be admixed with water or other diluent prior to administration to the animal.

### REFERENCES

Bieganowski, P. and C. Brenner, 2004. Discoveries of nicotinamide riboside as a nutrient and conserved NRK genes establish a Preiss-Handler independent route to NAD+ in fungi and humans. Cell. 117(4): 495-502.

Canto, C., K. J. Menzies, and J. Auwerx, 2015. NAD(+) Metabolism and the Control of Energy Homeostasis: A Balancing Act between Mitochondria and the Nucleus. Cell Metab. 22(1): 31-53.

Chambon, P., J. D. Weill, and P. Mandel, 1963. Nicotinamide mononucleotide activation of new DNA-dependent polyadenylic acid synthesizing nuclear enzyme. Biochem Biophys Res Commun. 1139-43.

Chen, L. K., et al. (2014). Sarcopenia in Asia: consensus report of the Asian Working Group for Sarcopenia. Journal of the American Medical Directors Association 15, 95-101.

Clark R V, Walker A C, O'Connor-Semmes R L, Leonard M S, Miller R R, Stimpson S A, Turner S M, Ravussin E, Cefalu W T, Hellerstein M K, Evans W J (1985). Total body skeletal muscle mass: estimation by creatine (methyl-d3) dilution in humans. J Appl Physiol. June 15; 116(12):1605-13.

Cruz-Jentoft, A. J., Baeyens, J. P., Bauer, J. M., Boirie, Y., Cederholm, T., Landi, F., Martin, F. C., Michel, J. P., Rolland, Y., Schneider, S. M., et al. (2010). Sarcopenia: European consensus on definition and diagnosis: Report of the European Working Group on Sarcopenia in Older People. Age Ageing 39, 412-423.

Fearon et al. (2011) Definition and classification of cancer cachexia: an international consensus. Lancet Oncology,12, 489-495.

Giroud-Gerbetant, J. et al. (2019) A reduced form of nicotinamide riboside defines a new pathway for synthesis of NAD+and acts as an orally bioavailable NAD+ precursor, Molecular Metabolism, Vol. 30, pp.192-202.

Imai, S., C. M. Armstrong, M. Kaeberlein, and L. Guarente, 2000. Transcriptional silencing and longevity protein Sir2 is an NAD-dependent histone deacetylase. Nature. 403(6771): 795-800.

Lee, H. C. and R. Aarhus, 1991. ADP-ribosyl cyclase: an enzyme that cyclizes NAD+ into a calcium-mobilizing metabolite. Cell Regul. 2(3): 203-9.

Makarov, M. and M. Migaud, 2019. Syntheses and chemical properties of β-nicotinamide riboside and its analogues and derivatives. Beilstein J. Org. Chem. 15: 401-430.

Matasic, D S, Brenner C, and London, B. 2018. Emerging potential benefits of modulating NAD+ metabolism in cardiovascular disease. Review: Mining Natural Products for Cardiovascular Benefits, April 2018.

Studenski S A, Peters K W, Alley D E, Cawthon P M, McLean R R, Harris T B, Ferrucci L, Guralnik J M, Fragala M S, Kenny A M, Kiel D P, Kritchevsky S B, Shardell M D, Dam T T, Vassileva M T (2014). The FNIH sarcopenia project: rationale, study description, conference recommendations, and final estimates. J Gerontol A Biol Sci Med Sci. 69(5), 547-558.

Stimpson S A, Leonard M S, Clifton L G, Poole J C, Turner S M, Shearer T W, Remlinger K S, Clark R V, Hellerstein M K, Evans W J. (2013) Longitudinal changes in total body creatine pool size and skeletal muscle mass using the D3-creatine dilution method. J Cachexia Sarcopenia Muscle. June 25.

### DESCRIPTION OF FIGURES

FIG. 1. Chemical structure of nicotinamide riboside in its oxidized (NR) and reduced (NRH) forms

1: 1-b-D-ribofuranosyl-3-pyridinecarboxamide salt

2: 1,4-dihydro-1-b-D-ribofuranosyl-3-pyridinecarboxamide

3: 1,2-dihydro-1-b-D-ribofuranosyl-3-pyridinecarboxamide

4: 1,6-dihydro-1-b-D-ribofuranosyl-3-pyridinecarboxamide

X⁻: anion (e.g. triflate)

FIG. 2. Dose-response experiments revealed that NRH could significantly increase

NAD+better than NR

Starting at levels at a concentration of 10 μM, NRH achieved similar increases in intracellular NAD+ levels to those reached with NR at 50-fold higher concentrations. NRH achieved maximal effects on NAD+ synthesis around the millimolar range, managing to increase intracellular NAD+ levels by more than 10-fold.

FIG. 3. NHR acts rapidly after 5 minutes from treatment.

NRH actions were also extremely fast, as significant increases in NAD+ levels were observed within 5 minutes after NRH treatment. Peak levels of NAD+ were achieved between 45 minutes and 1 h after treatment.

FIG. 4. NRH leads to NAD+ biosynthesis through an adenosine kinase dependent path.

AML12 cells were treated with an adenosine kinase inhibitor (5-IT; 10 mM) for 1 hour prior to NRH treatment at the doses indicated. Then, 1 hour later, acidic extracts were obtained to measure NAD⁺ levels. All values in the figure are expressed as mean+/−SEM of 3 independent experiments. * indicates statistical difference at p<0.05 vs. the respective vehicle treated group.

FIG. 5. NRH is an orally active NAD+ precursor in mice.

8 week-old C57BI/6NTac mice were orally gavaged with either saline (as vehicle), NR (500 mg/kg) or NRH (500 mg/kg). After 1 hour, liver, skeletal muscle and kidney NAD⁺ levels were evaluated. All results are expressed as mean+/−SEM of n=5 mice per group. * indicates statistical difference at p<0.05 vs. vs. saline-treated mice. # indicates statistical difference at p<0.05 vs. NR treated mice.

FIG. 6. NRH is found intact in mice tissues after administration.

8 week-old C57BI/6NTac mice were orally gavaged with either saline (as vehicle), and NRH (250 mg/kg). After 2 hours, liver, skeletal muscle and kidney NRH levels were evaluated. All results are expressed as mean+/−SEM of n=4 mice per group, as areas under the signal by LC-MS analysis, corrected by total protein amount of tissue.

FIG. 7. NRH is found intact in heart after oral administration.

8 week-old C57BI/6NTac mice were orally gavaged with either saline (as vehicle), and an stable isotope-labelled NRH (250 mg/kg). After 2 hours, cardiac NRH levels were evaluated. All results are expressed as mean+/−SEM of n=4 mice per group, as areas under the signal by LC-MS analysis, corrected by total protein amount of tissue.

FIG. 8. NRH rescues Doxorubicin-induced toxicity in Zebrafish

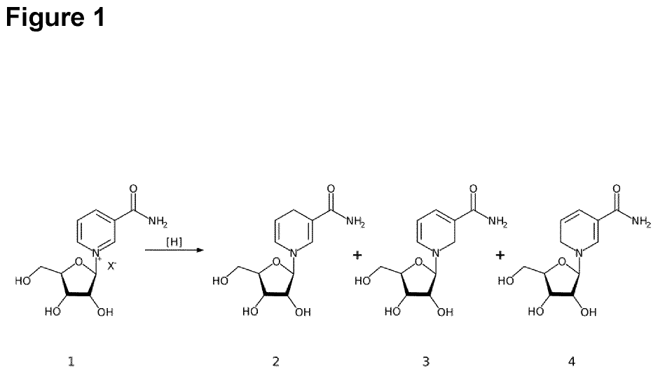

1 day post fertilization zebrafish embryos were placed in 24 well plates and treated with Doxorubicin (25 uM), either alone or in combination with NRH (500 uM). Untreated group was exposed to DMSO (0.05%). Health of the larvae was monitored frequently, and dead larvae were scored overtime, up to 60 h treatment. Results are expressed as percent of survival of n=60 embryos per group. Statistics represented were calculated by pairwise comparison using log-rank test and p-value adjustment was performed by Bonferroni correction. p-Value for median survival of untreated vs Doxorubicin <0.0001, p-value Doxorubicin vs Doxorubicin+NRH <0.0001.

FIG. 9. NRH protects against ethanol-induced cardiac edema in Zebrafish.

Phenotypic scoring of cardiac edema severity in zebrafish larvae at 3 days post fertilization exposed to 2% Ethanol during 20 h with or without NRH (500 uM) co-treatment (untreated, n=60; Ethanol, n=73; Ethanol +NRH, n=72). Statistics represented were calculated by non-parametric Chi-square test without p-value adjustment. **** p-Value<0.0001, * p-Value=0.0275.

FIG. 10. Dihydronicotinamide Riboside (NRH) protects against oxidative stress-induced cardiomyocyte cell death.

H9C2 cardiomyocytes were differentiated for 6 days and then were treated with hydrogen peroxide (H₂O₂; at either 0.25 mM or 0.5 mM) for 24 hrs in the presence or absence of NRH (0.1 mM). PBS was used as vehicle for NRH. After 24 hrs, cell viability was evaluated by lumiscence using a commercial kit (Cell Title-Glow; Promega). All results are expressed as meand+/−SEM of n=6 experiments. * indicates P<0.05 vs. the respective vehicle treated group.

### EXAMPLES

### Example 1: Synthesis of the Reduced Form of Nicotinamide Riboside (NRH)

Reduced nicotinamide riboside (NRH) was obtained from NR (1) by reduction of pyridinium salts (for example, triflate) to dihydropyridines (1,2-, 1,4-, and 1,6-dihydropyridines) as shown below

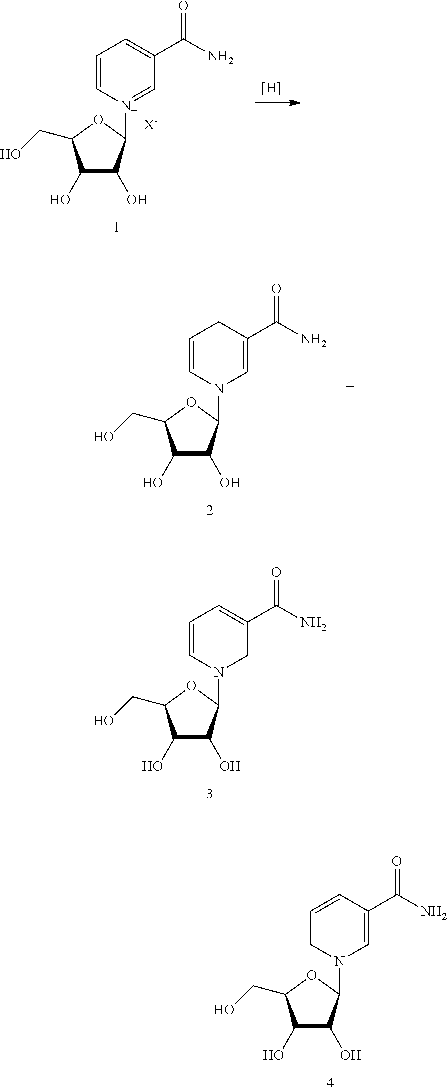

1: 1-b-D-ribofuranosyl-3-pyridinecarboxamide salt

2: 1,4-dihydro-1-beta-D-ribofuranosyl-3-pyridinecarboxamide

3: 1,2-dihydro-1-beta-D-ribofuranosyl-3-pyridinecarboxamide

4: 1,6-dihydro-1-beta-D-ribofuranosyl-3-pyridinecarboxamide

X⁻: anion (e.g. triflate)

Sodium borohydride (NaBH₄) and sodium dithionite (Na₂S₂O₄) were used as reducing agents for N-substituted pyridinium derivatives. Regioselectivity of reducing agents differ, leading to either only one dihydropyridine or a mixture of all 3 isomers in different proportions (2,3,4).

Dithionate reduction of pyridinium salts, carrying electron withdrawing substituents in positions 3 and 5, yielded almost exclusively 1,4-dihydropyridine products. The reduction was made in mild conditions (e.g. in aqueous sodium bicarbonate or potassium phosphate dibasic medium), due to instability of the reduced products in acidic media. To perform the reduction, hydroxyl groups in the ribofuranose moiety were protected with either benzyl or acetyl substituents. Deprotection was then be done by sodium hydroxide in methanol under ball mill conditions, after reduction.

### Example 2: Measurement of NRH and other NAD+ Related Metabolites in Biological Samples

Levels of NRH and other NAD-related metabolites in biological samples were obtained by using a cold liquid-liquid extraction using a mixture of methanol:water:chloroform in 5:3:5 (v/v), from which the polar phase was recovered for for hydrophilic interaction ultra-high performance liquid chromatography mass spectrometry (UHPLC-MS) analysis. The UHPLC consisted of a binary pump, a cooled autosampler, and a column oven (DIONEX Ultimate 3000 UHPLC+ Focused, Thermo Scientific), connected to a triple quadrupole spectrometer (TSQ Vantage, Thermo Scientific) equipped with a heated electrospray ionisation (H-ESI) source. Of each sample, 2 μL were injected into the analytical column (2.1 mm×150 mm, 5 μm pore size, 200 Å HILICON iHILIC®-Fusion(P)), guarded by a pre-column (2.1 mm×20 mm, 200 Å HILICON iHILIC®-Fusion(P) Guard Kit) operating at 35° C. The mobile phase (10 mM ammonium acetate at pH 9, A, and acetonitrile, B) was pumped at 0.25 mL/min flow rate over a linear gradient of decreasing organic solvent (0.5-16 min, 90-25% B), followed by re-equilibration for a total run time of 30 min. The MS operated in positive mode at 3500 V with multiple reaction monitoring (MRM). The software Xcalibur v4.1.31.9 (Thermo Scientific) was used for instrument control, data acquisition and processing. Retention time and mass detection was confirmed by authentic standards.

Structure elucidation of the used NRH for biological studies was confirmed by nuclear magnetic resonance (NMR).

### Example 3: NRH is a Potent NAD+ Precursor

AML12 hepatocytes were treated with NRH, and it was observed that the ability of NRH to increase intracellular NAD+ was superior to that of NR.

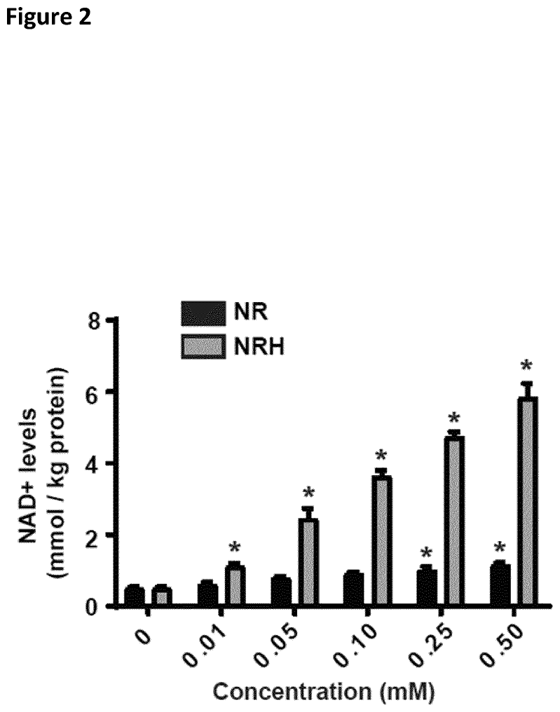

Dose-response experiments revealed that NRH could significantly increase NAD+ levels at a concentration of 10 μM (FIG. 2). Even at such relatively low dose, NRH achieved similar increases in intracellular NAD+ levels to those reached with NR at 50-fold higher concentrations. NRH achieved maximal effects on NAD+ synthesis around the millimolar range, managing to increase intracellular NAD+ levels by more than 10-fold.

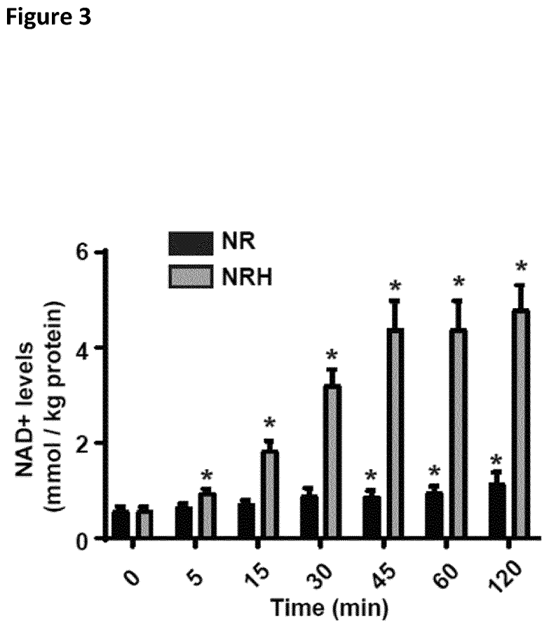

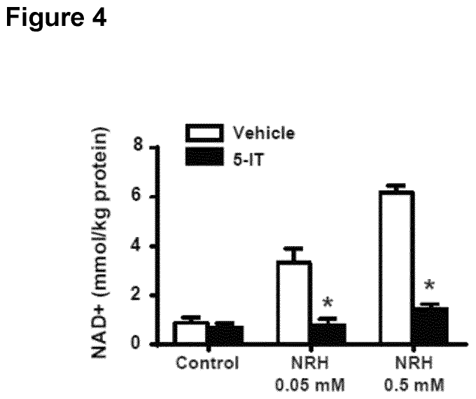

NRH actions were also extremely fast (FIG. 3), as significant increases in NAD+ levels were observed within 5 minutes after NRH treatment. Peak levels of NAD+ were achieved between 45 minutes and 1 h after treatment, as also occurred with NR.

The ability of NRH to potently increase NAD+ was tested as well in other cell type models. NRH treatment highly elevated NAD+ levels in C2C12 myotubes, INS1-cells and 3T3 fibroblasts, supporting the notion that NRH metabolism is widely conserved among different cell types.

### Example 4: Pathway of NRH-Induced NAD+ Synthesis

A path in which NRH would be converted to NMNH, then to NADH and this would be finally oxidized to NAD+. Accordingly, NRH and NMNH could be detected intracellularly 5 minutes after NRH, but not NR, treatment. Interestingly, NRH treatment also led to an increase in intracellular NR and NMN, greater than that triggered by NR itself, opening the possibility that NRH could synthesize NAD+ by being oxidized to NR, using then the canonical NRK/NMNAT path.

In order to understand the exact path by which NRH synthesizes NAD+, we initially evaluated whether NRH, could be transported into the cell by equilibrative nucleoside transporters (ENTs). Confirming this possibility, NRH largely lost its capacity as an extracellular NAD+ precursor in the presence of an agent blocking ENT-mediated transport, such as S-(4-nitrobenzyl)-6-thioinosine (NBTI). Nevertheless, a substantial action of NRH remained even after ENT blockage, suggesting that NRH might be able to enter the cell through additional transporters.

The action of NRH was also NAMPT-independent, based on experiments using FK866, a NAMPT inhibitor. If NRH led to NAD+ synthesis via the formation of NMNH, this hypothetical path would require the phosphorylation of NRH into NMNH. Given the essential and rate-limiting role of NRK1 in NR phosphorylation, we wondered whether the ability of NRH to boost NAD+ levels was NRK1 dependent. To answer this question, we evaluated NRH action in primary hepatocytes from either control or NRK1 knockout (NRK1KO) mice. While after 1 hour of treatment NR failed to increase NAD+ levels in NRK1KO derived primary hepatocytes, NRH action was not affected by NRK1 deficiency. These results indicate that NRH action is NRK1 independent. Further, they rule out the possibility that NRH-induced NAD+ transport is driven by NRH oxidation into NR.

Considering the molecular structure of NRH, we reasoned that an alternative nucleoside kinase could be responsible for the phosphorylation of NRH. Confirming this expectation, the adenosine kinase (AK) inhibitor 5-iodotubercidin (5-IT) fully ablated the action of NRH. The role of AK in NRH-mediated NAD+ synthesis was confirmed using a second, structurally different, AK inhibitor, ABT-702. Metabolomic analyses further confirmed that upon inhibition of AK, the generation of NMNH, NADH and NAD+ was fully blunted, even if NRH was effectively entering the cell. Interestingly, 5-IT treatment also prevented the formation of NR and NMN after NRH treatment.

This indicates that the occurrence of NR after NRH treatment cannot be attributed simply to direct NRH intracellular oxidation to NR. As a whole, these experiments depict adenosine kinase as the enzymatic activity catalyzing the conversion of NRH into NMNH, initiating this way the transformation into NAD+.

As a follow-up step, NMNAT enzymes could catalyze the transition from NMNH to NADH. Accordingly, the use of gallotannin as a NMNAT inhibitor largely compromised NAD+ synthesis after NRH treatment. Yet, part of the NRH action remained after gallotannin treatment when NRH was used at maximal doses. However, NRH action was totally blocked by gallotannin at submaximal doses, suggesting that the remaining effect at 0.5 mM could be attributed to incomplete inhibition of NMNAT activity by gallotannin. Altogether, these results indicate that adenosine kinase and NMNATs vertebrate the path by which NRH leads to NAD+synthesis via NADH.

### Example 5: NRH is Detectable in Circulation After IP Injection

NR degradation to NAM has been proposed as a limitation for its pharmacological efficacy. To evaluate whether NRH was also susceptible to degradation to NAM, we spiked NRH or NR in isolated mouse plasma. After 2 h of incubation, NR levels decayed in plasma, in parallel to an increase in NAM. In contrast, NAM was not generated from NRH, as its levels remained stable during the 2 h test. We also tested the stability of NRH in other matrixes. Given our previous experiments in cultured cells, we verified that NRH did not degrade to NAM in FBS supplemented media, as occurs with NR. Finally, we also certified NRH stability in water (pH=7, at room temperature) for 48 h.

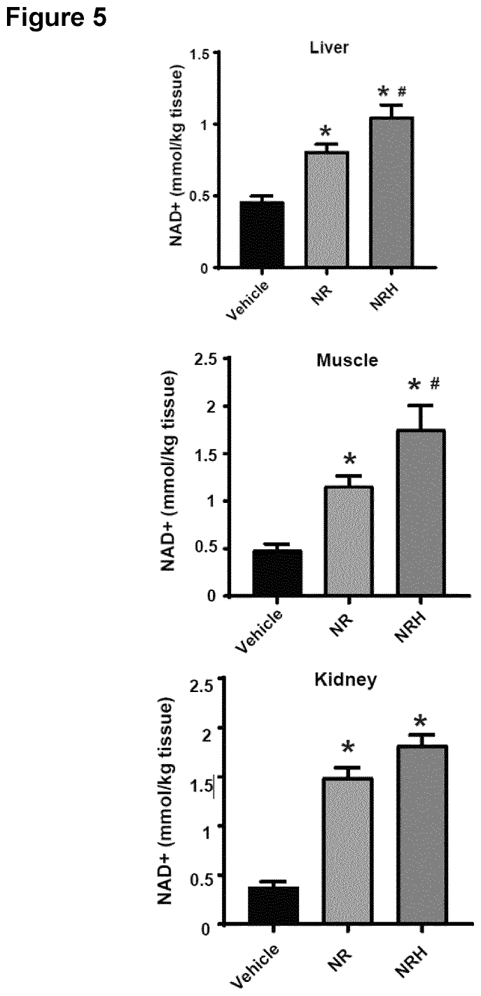

The above results prompted us to test whether NRH could act as an effective NAD+ precursor in vivo. For this, we first intraperitoneally (IP) injected mice with either NR or NRH (500 mg/kg). After 1 h, both compounds increased NAD+ levels in liver (FIG. 5), muscle and kidney. As expected, NAM levels were highly increased in circulation upon NR administration, while only a very mild increase was observed with NRH. Importantly, NRH was detectable in circulation after IP injection.

To our surprise, NR was detectable in circulation after NRH treatment at much higher levels than those detected after NR injection itself. Given that NRH incubation in isolated plasma did not lead to NR production, the appearance of NR might be consequent to intracellular production and release to circulation. Similarly, the residual appearance of NAM after NRH treatment might be explained by the degradation of released NR or by the release of intracellular NAM as a product of NAD+ degradation, as NRH did not significantly alter NAM levels when incubated in isolated plasma.

### Example 6: NRH is Detectable After Oral Administration as an Orally Bioavailable NAD+ Precursor that Overcomes Direct Degradation in Plasma

Oral administration of NRH led to very similar results to those observed after IP administration. First, NRH had a more potent effect on hepatic NAD+ levels than NR. NRH was detectable in plasma 1 h after oral administration. In contrast, NR levels were undetectable at 1 h after NR administration. As expected, NR treatment led to large increases in circulating NAM, which where ˜4-fold higher than those observed after NRH treatment. Quantification measurements revealed that after oral gavage, NRH concentration in plasma reached 11.16±1.74 micromolar, which is enough to effectively drive NAD+ synthesis. These results illustrate that NRH is a potent orally bioavailable NAD+ precursor that overcomes direct degradation to NAM in plasma.

### Example 7: NRH is Found Intact in Liver, Kidney and Muscle After Oral Administration

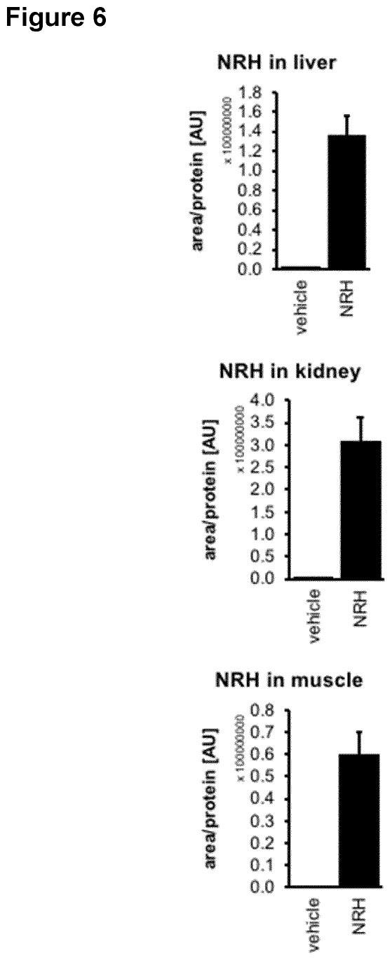

NRH is not only found in circulation but it was also found intact, in high levels, in mice liver, kidney and muscle 2 hours after gavage (FIG. 6). This indicates that oral administration of NRH allows for efficient biodistribution in target tissues.

### Example 8: NRH is Found Intact in Heart After Oral Administration

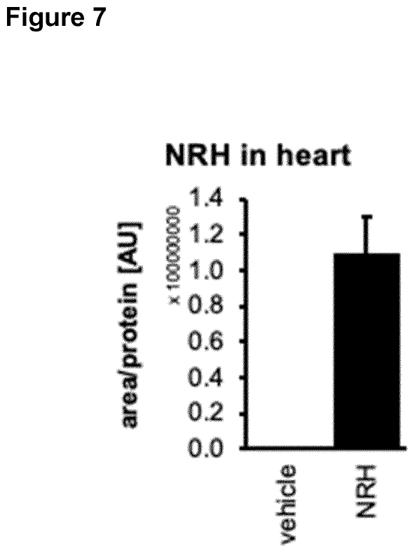

After oral administration, NRH is found, among various tissues, in mice heart at high levels 2 hours after gavage (FIG. 7). This indicates that oral administration of NRH allows for efficient biodistribution in the heart.

### Example 9: Cardioprotective Effects of NRH

Effect on Cardiac Toxicity Rescue: Treatment with Doxorubicin

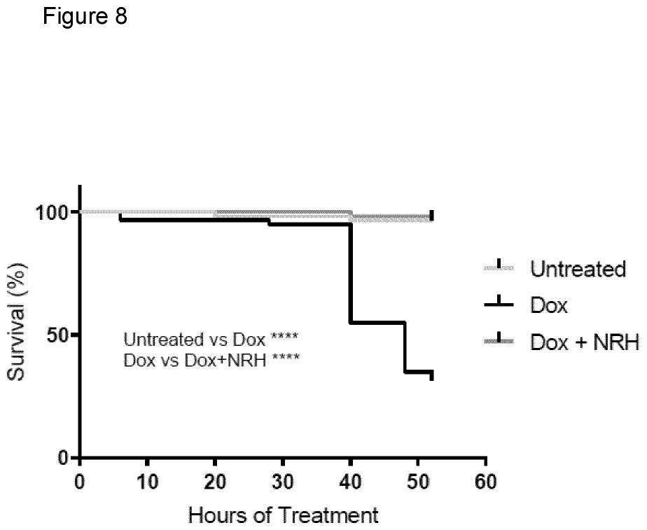

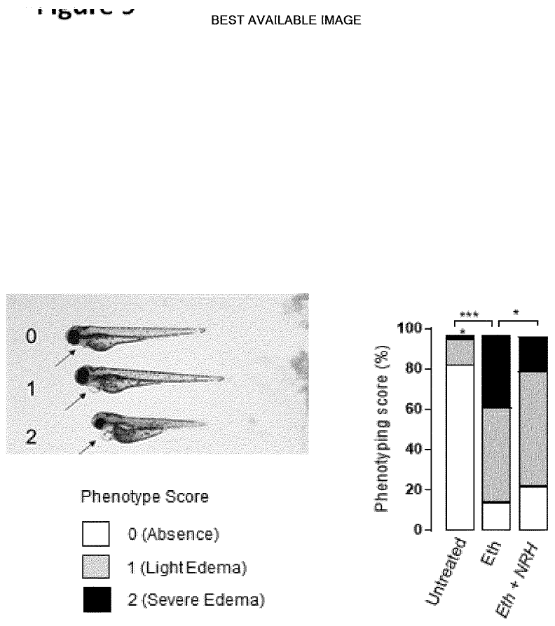

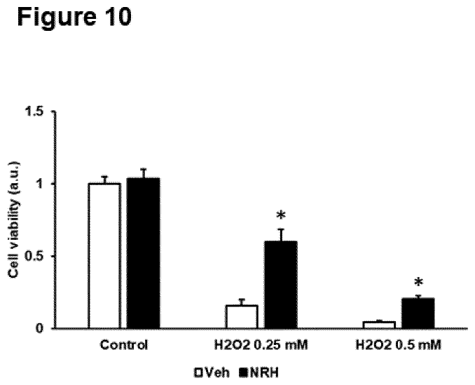

Doxorubicin is a chemotherapeutic agent that is well established to be cardiotoxic. This drug has also been described to induce cardiomyopathy in zebrafish larvae. 1 day post fertilization zebrafish embryos were placed in 24 well plates and treated with Doxorubicin (25 uM), either alone or in combination with NRH (500 uM). Untreated group was exposed to DMSO (0.05%). Health of the larvae was monitored frequently, and dead larvae were scored overtime, up to 60 h treatment. Results are expressed as percent of survival of n=60 embryos per group. Results in FIG. 8 showed that NRH rescues doxorubicin induced toxicity in zebrafish larvae indicating a cardioprotective effect of NRH.

Effect on Heart Edema: Treatment with 2% Ethanol

Phenotypic scoring of cardiac edema severity in zebrafish larvae at 3 days post fertilization exposed to 2% Ethanol during 20 h with or without NRH (500 uM) co-treatment (untreated, n=60; Ethanol, n=73; Ethanol+NRH, n=72). Results are presented in FIG. 9. This indicates NRH protects against ethanol-induced cardiac edema in Zebrafish.

### Example 10. Dihydronicotinamide Riboside (NRH) Protects Against Oxidative Stress-Induced Cardiomyocyte Cell Death

H9C2 cardiomyocytes were differentiated for 6 days and then were treated with hydrogen peroxide (H₂O₂; at either 0.25 mM or 0.5 mM) for 24 hours in the presence or absence of NRH (0.1 mM). PBS was used as vehicle for NRH. After 24 hours, cell viability was evaluated by lumiscence using a commercial kit (Cell Title-Glow; Promega). Results are presented in FIG. 10. This indicates that NRH protects against oxidative stress-induced cardiomyocyte cell death.

1. A method of increasing intracellular NAD+ in a subject comprising delivering to the subject in need thereof, an effective unit dose form of reduced nicotinamide to prevent and/or treat cardiovascular diseases or conditions.

2. Method according to claim 1 wherein said reduced nicotinamide riboside is selected from the group consisting of:
(i) 1,4-dihydro-1-beta-D-ribofuranosyl-3-pyridinecarboxamide;
(ii) 1,2-dihydro-1-beta-D-ribofuranosyl-3-pyridinecarboxamide; and
(iii) 1,6-dihydro-1-beta-D-ribofuranosyl-3-pyridinecarboxamide .

3. Method according to claim 1 wherein the reduced nicotinamide riboside is 1,4-dihydro-1-beta-D-ribofuranosyl-3-pyridinecarboxamide.

4. Method according to claim 1 wherein said dose is used to prevent and/or treat cardiovascular diseases or conditions.

5. Method according to claim 4 wherein said dose consists essentially of reduced nicotinamide riboside without other NAD+ precursors to prevent and/or treat cardiovascular diseases or conditions.

6. Method according to claim 1 to maintain or improve cardiovascular function in a subject.

7. Method according to claim 1 to enhance recovery of the cardiovascular system, especially cardiac tissue after injury or surgery.

8. Method according to claim 1 wherein said dose is a nutritional composition selected from the group consisting of a: food and beverage product.

9. Method according to claim 1 wherein the cardiovascular disease and/or condition is selected from the group consisting of: Coronary heart disease; Cerebrovascular disease; Peripheral arterial disease; Rheumatic heart disease and other inflammatory heart conditions; Congenital heart disease; and Deep vein thrombosis and pulmonary embolism.

10. Method to prevent and/or treat cardiovascular diseases or conditions selected from the group consisting of: Coronary heart disease; Cerebrovascular disease; Peripheral arterial disease; Rheumatic heart disease and other inflammatory heart conditions; Congenital heart disease; and Deep vein thrombosis and embolism in a subject mammal comprising delivering to the mammal in need of such treatment an effective amount of reduced nicotinamide riboside.

11. Method for increasing intracellular NADH in a subject mammal, comprising delivering to the mammal in need of such treatment an effective amount of reduced nicotinamide riboside to enhance recovery after cardiovascular injury or surgery.

12. Method according to claim 10 for preventing and/or treating a cardiovascular disease or conditions in a subject in need comprising the steps of:
i) providing the subject a composition consisting essentially of reduced nicotinamide riboside and
ii) administering the composition to said subject.

13. Method according to claim 10 wherein the subject is selected from the group consisting of: human, dog, cat, cow, horse, pig, and sheep.

14. Method according to claim 13 wherein the subject is a human.
